(19) United States
(12) Patent Application Publication
(10) Pub. No.: US 2006/0165784 A1
(43) Pub. Date: Jul. 27, 2006

(54) Calcium supplement

(76) Inventor: Jin R. Zhao, Pasadena, CA (US)

Correspondence Address: Jin R. ZHAO, M.D.;c/o Michael R. Doram Esq., Suite 202, 1028 North Lake Avenue, Pasadena, CA, 91104, US

(21) Appl. No.: 11/081,780

(22) Filed: Mar. 17, 2005

Related U.S. Application Data
(63) Continuation-in-part of application No. 10/160,957, filed on May 31, 2002, now abandoned.
Application No. 10/160,957 is a continuation-in-part of application No. 09/748,775, filed on Dec. 27, 2000, now abandoned.
Application No. 09/748,775 is a continuation-in-part of application No. 08/852,238, filed on May 6, 1997, now abandoned.

(51) Int. Cl. A61K 9/20; A61K 33/10

(52) U.S. Cl. 424/464; 424/687

(57) ABSTRACT

A dissoluble oral tablet of calcium supplement and the method of making the product are provided. This calcium supplement comprises an exposure area and a coating covered area. The surface ratio between the exposure area and the coating covered area is about 1:1 to 1:12. The exposure area can be a hole, an opening, or their combination. The preferred samples contain about CaCO3 200-600 mg with MgCL2 50-150 mg or MgSO4 50-100 mg. The favorable CaCO3 tablet reaction with stomach acid is: CaCO3+2HCL=CaCL2+H2CO3=Ca⁺⁺+2CL⁻+H2O+CO2 ↑. The CO2 is the natural bubble broker. The stomach HCL is the natural CaCL2 maker.

### RELATED APPLICATIONS

This is a continuation-in-part application of copending application Ser. No. 10/160,957 filed May 31, 2002, which is a continuation-in-part of Ser. No. 09/748,775 filed on Dec. 27, 2000, now abandoned and copending application Ser. No 09/240,832 filed on Jan. 9, 2001, now U.S. Pat. No. 6,171,635B1, which is a continuation-in-part of application Ser. No. 08/852,238 filed on May 6, 1997, now abandoned.

### FIELD OF THE INVENTION

The present invention relates to oral tablet, especially to a soluble calcium tablet.

### BACKGROUND OF THE DISCLOSURE

Calcium supplement is widely used for lowing a person's risk of developing osteoporosis and colon carcinoma. Calcium might also ease some Alzheimer's disease, diabetes type II, and premenstrual syndrome. Since calcium gluconate, calcium citrate, calcium chloride, and calcium lactate contain lower content of calcium, people may have to take a larger tablet to get the same dose of calcium. For example, supplement form in calcium citrate contains 21% elemental calcium, whereas calcium carbonate contains 40% elemental calcium. Besides, strong acids have a tendency to reduce bone density. Thus, most calcium pills in the market are in the form of CaCO3, e.g., coral calcium.

Comparing to CaCL2, CaCO3 requires extra stomach acid for better absorption. Since most foods leave the stomach within 15 minutes-2.5 hours, CaCO3 supplement had better be dissolved in the stomach before its turn for leaving. Besides, current coating is made of water-insoluble material, e.g. wax, polymer, glue, to form a continuous hard coating. If a pill causes the stomach upset or ulcer, its exposure should be delayed until its passing the stomach. However, CaCO3 does not irritate the stomach, and consequently, the general coating and making method are deviated from the goal of calcium supplement, especially CaCO3. Furthermore, the coating takes about 2-25% by weight of a tablet and some coating is so hard that its sharp edge may cut the mucous once the pill is broken, which should be avoided.

At first, the disclosure tries to help CaCO3 getting dissolved in the acidic stomach before going to the alkaline environment in the intestine. Secondly, the disclosure intends to make it happen in a natural way without expensive process or contamination. The 3rd main aim is the health and physical cooperation between calcium and magnesium.

### BRIEF DESCRIPTION OF THE DRAWINGS

FIG. 1 shows the cross section view of a CaCO3 pill. The central area is punched into a hole. The continuous double line is the coating whereas the break line is the hole.

FIG. 2 shows the overview of FIG. 1.

FIG. 3 shows the cross section view of a calcium tablet with its both sides covered by coating (double line), leaving the out circular area being naked (break line) to environment.

### DETAILED DESCRIPTION

To solve the above matter, a novel approach is taken as the following:

1) Some sample of CaCO3 tablet can be soaked in water for 24 hours without being broken. Most food can only stay in the stomach for 15 minutes to 2.5 hours. Although the tablet can be broken in the intestine, there is no HCL to dissolve it. The current paradox will cause not only low dissolution and absorption of calcium supplement, but also side effects, e.g. constipation, which worse hypertension and diabetes type II. Therefore, the continuous coating of current calcium supplement has to be broken.

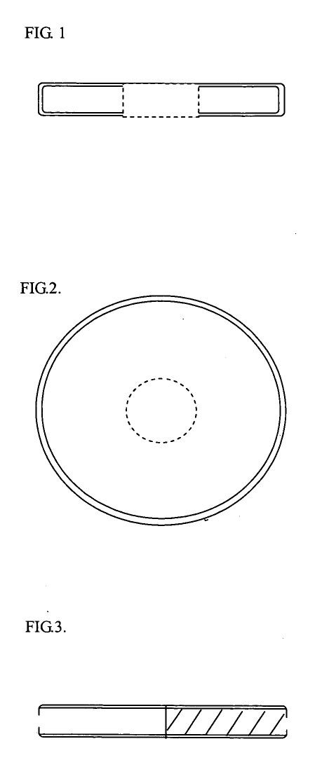

According to FIG. 1, a dissoluble oral tablet comprises an exposure area and a covered area; wherein the surface ratio between the exposure area and the covered area being approximately 1:1 to 1:12.

2) For example, a dissoluble oral tablet of calcium supplement comprises an exposure area being naked and a covered area with coating; wherein the exposure area comprising a hole to both sides, an opening to one side, or a combination thereof; wherein the surface ratio between the exposure area and the covered area being approximately 1:1 to 1:12, preferably, 1:1 to 1:10.

3) Furthermore, this dissoluble oral tablet is a calcium supplement of CaCO3.

4) Particularly, the dissoluble oral tablet contains CaCO3 200-600 mg and MgSO4 50-100 mg or MgCL2 50-150mg. Magnesium may not only cooperate with calcium to reduce osteoporosis but also ease hypertension.

5) This dissoluble oral tablet contains a naked area with its inside being opened to outside.

6) The dissoluble oral tablet contains a coating that is made of water-soluble material.

7) The method of making a dissoluble oral tablet comprising the step of making an exposure area on an oral tablet; wherein the surface ratio between the exposure area and the covered area being approximately 1:1 to 1:12.

8) For example, the method of making a dissoluble oral table contains the step of punching a hole in the central area of a coated oral tablet.

9) Furthermore, the method comprises the step of leaving an exposure area on an oral tablet with other area being covered by a water-soluble coating. This exposure area is naked to outside environment so that a reaction will help the dissolution of CaCO3 as the following:
CaCO3+2HCL=CaCL2+H2CO3=Ca⁺⁺+2CL⁻+H2O+CO2↑

10) This CO2 itself is the best natural bubble breaker for the dissolution of CaCO3. The HCL in the stomach is the best natural maker of CaCL2. The method to make a favorable cooperation and result to happen is to leave an exposure area so that it can have a chance to occur. This dissoluble oral tablet can be illustrated by examples as the following:

### EXAMPLE 1

The dissoluble oral tablet contains CaCO3 200 mg and MgCL2 75 mg once to twice daily for reducing calcium intake insufficient related osteoporosis. A hole is punched at the center of the tablet. The diameter of the hole is about 3 mm. Magnesium is the best natural cooperative element to help calcium deposit/stay within the bone matrix. Plus, magnesium may reduce calcium related kidney stone. This pill is made in a small dose and size to flatten the postgrandial blood calcium levels to reduce side effects. Thus, the daily calcium supplement can be provided in a natural and smooth way. Preferably, the coating is made of water-soluble material, e.g. sugar, starch, water-soluble fiber or amino acid. Color agent, e.g. aluminum blue, TiO2 whitener, is rather not used.

The range of calcium supplement daily is about 300-800 mg to fill the gap caused by food insufficient, up to 800-1200 mg for bone fracture. Preferably calcium 300-600 mg with magnesium 50-350 mg, especially 50-200 mg, up to 350 mg for hypertension. Usually an effective minimum dose is safer than a full dose in a long-term.

### EXAMPLE 2

The dissoluble oral tablet contains CaCO3 300 mg and MgSO4 150 mg with ZnSO4 2.5 mg, twice per day for helping acute bone fracture. Coating covers the up and low surfaces of the tablet and leaves most outside circle of the tablet being naked as FIG. 3.

Although a preferred embodiment of the invention has been specifically illustrated and described herein, it is to be understood that variations may be made without departing from the spirit and scope of the invention, as defined by the appended claims.

What is claimed is:

1. A dissoluble oral tablet comprising an exposure area and a covered area; wherein said exposure area comprising a hole, an opening, or a combination thereof; wherein the surface ratio between the exposure area and said covered area being approximately 1:1 to 1:12.

2. A dissoluble oral tablet of calcium supplement comprising an exposure area being naked and a covered area with coating; wherein said exposure area comprising a hole to both sides, an opening to one side, or a combination thereof; wherein the surface ratio between the exposure area and said covered area being approximately 1:1 to 1:12.

3. The dissoluble oral tablet according to claim 1, wherein said dissoluble oral tablet comprising CaCO3.

4. The dissoluble oral tablet according to claim 3 comprising CaCO3 200-600 mg and MgSO4 50-100 mg.

5. The dissoluble oral tablet according to claim 3 comprising CaCO3 200-600 mg and MgCL2 50-150 mg.

6. The dissoluble oral tablet according to claim 1, 2, 3, 4, or 5, wherein said dissoluble oral tablet comprising a naked area with its inside being opened to outside.

7. The dissoluble oral tablet according to claim 1, 2, 3, 4, or 5, wherein said dissoluble oral tablet comprising a coating that is made of water-soluble material.

8. A method of making a dissoluble oral tablet comprising the step of making an exposure hole on an oral tablet; wherein the surface ratio between the exposure area and said covered area being approximately 1:1 to 1:12.

9. The method of making a dissoluble oral table according to claim 1 comprising the step of punching a hole in the central area of a coated oral tablet.

10. The method of making a dissoluble oral tablet according to claim 1 comprising the step of leaving an exposure area on an oral tablet with other area being covered by a water-soluble coating, wherein said exposure area being naked to outside.
